(19) United States
(12) Patent Application Publication
(10) Pub. No.: US 2026/0159487 A1
(43) Pub. Date: Jun. 11, 2026

(54) FORMULATION FOR AROMATIZING A FOOD PRODUCT

(71) Applicant: DÔHLER GMBH, Darmstadt (DE)

(72) Inventors: Hadeel ALJENDY, Damstadt (DE); Agostino BIAFORA, Damstadt (DE); Daniel KODURA, Damstadt (DE); Christian RÔSNER, Damstadt (DE)

(73) Assignee: DÔHLER GMBH, Darmstadt (DE)

(21) Appl. No.: 19/127,157

(22) PCT Filed: Nov. 13, 2023
(86) PCT No.: PCT/EP2023/081648

§ 371 Date: May 5, 2025

(30) Foreign Application Priority Data
Dec. 6, 2022 (DE) 102022 132 286.6

(51) Int. Cl. C07D 241/38; C07D 241/12

(52) U.S. Cl. CPC C07D 241/38; C07D 241/12

(57) ABSTRACT

The present invention relates to a process for producing a formulation for aromatizing a product, in particular a food product, a semi-luxury food product, cosmetic or pharmaceutical product, and dietary supplement, which preferably comprises at least one (alkyl) pyrazine according to formula (Ia),

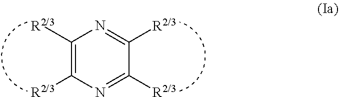

and/or at least one (alkyl) pyrazine according to formula (Ib)

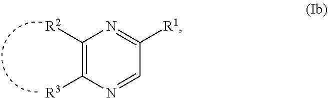

wherein R¹, R², and R³ represent the groups defined in the claims.

The invention relates to a formulation for aromatizing a food product, semi-luxury food product, cosmetic or pharmaceutical product, and dietary supplement, and to a process for producing such formulation.

It has been known that among pyrazines substituted with one or more alkyl or cycloalkyl groups, there are naturally occurring highly potent aroma compounds, some of which have a very low odor threshold. These alkyl pyrazines, such as 2-ethyl-3,5-dimethylpyrazine or 2,3-diethyl-5-methylpyrazine, have an earthy odor. Corylone pyrazine (also known as 5-methyl-6,7-dihydro-cyclopentapyrazine or nutty pyrazine) is a aromatizing compound with a strong odor. This odor can be described as sweet, nutty, roasted, toasty, earthy, as well as a cereal-, coffee-, and popcorn-like savory odor. Corylone pyrazine is found in the natural flavors of coffee and numerous nuts. In the flavor industry, this compound is used to enhance the scent of popcorn, for example.

It is known from the prior art that the addition of an alkyl-substituted pyrazine, alone (U.S. Pat. No. 3,579,353) or in combination with other compounds (GB1401096A), to food or delicatessen products leads to an improvement of the product flavor. U.S. Pat. No. 3,579,353 discloses three reactions for the production of alkyl-substituted pyrazines: diketone plus ethylenediamine; alkyl lithium plus dialkyl pyrazines; and sodium amide plus alkyl pyrazines.

Various approaches for the production of pyrazines have been described in the literature (Ong et al., Borneo Journal of Resource Science and Technology, 2017, 7 (2), pp. 60-75). Classical synthesis routes involve the use of various metal compounds. For example, pyrazines can be produced by condensation of 1,2-diaminoalkane with 1,2-dicarbonyl compounds, using copper (II) oxide and manganese oxide as oxidizing agents.

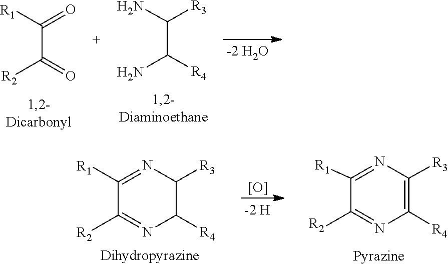

Pyrazines can also be obtained by condensation of two molecules of an α-amino ketone or an α-amino aldehyde. The resulting dihydropyrazines are converted into a pyrazine using an oxidizing agent, such as salts of divalent mercury:

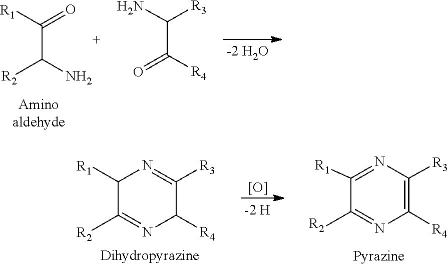

U.S. Pat. No. 4,097,478A discloses a process for preparing pyrazines in a gas phase contact reaction at 300° C. to 600° C. in the presence of a zinc-containing catalyst, using diols and diamines as starting compounds:

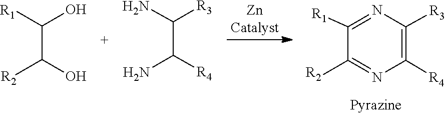

DE 695 18 206 T2 (Firmenich S. A.) discloses the use of two different hydroxy ketones for producing asymmetrically substituted alkyl pyrazines. When 1,3-dihydroxypropan-2-one is reacted with an acyloin of formula (III), substituted pyrazines such as those shown in the following diagram are obtained.

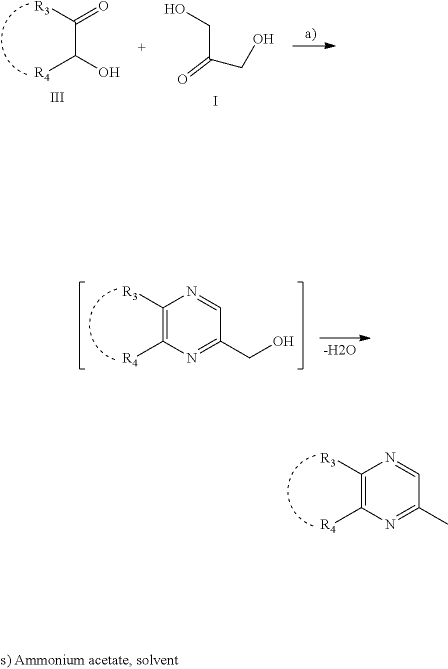

However, the hydroxy ketones and dihydroxy ketones used are relatively unstable and expensive.

Pyrazines can be produced through various synthesis routes, but all currently known processes have certain drawbacks. These include the use of solvents and metal compounds, some of which are highly toxic, as well as low yields of the target product and high reaction temperatures.

The toxic properties of catalysts and solvents can lead to environmental and health risks and must be considered in occupational health and safety both in the manufacture and the use of these catalysts. Complex and costly measures are required to ensure the necessary safety in the face of toxicological and health problems that primarily arise in the workplace. Furthermore, some of the catalysts used are very expensive compounds, which means that the purchase, disposal, or recycling of these catalysts can have a negative impact on the price of the final product.

Another way of obtaining pyrazines involves isolating these compounds from animal material using known separation techniques such as, e.g., steam distillation of cooked pork liver. It is known that pyrazines, such as alkyl pyrazines, acetyl pyrazines, cyclopentapyrazines and quinoxalines represent the largest group of aroma compounds in cooked pork liver (Mussinan C. J. and Walradt J. P. (1974) Volatile Constituents of Pressure Cooked Pork Liver, J. Agrc. Food Chem., 22, No. 5, 827-831). Pyrazines are among the so-called reaction flavors. These aroma-active compounds are formed when heated during cooking, frying and/or baking and are among the substances responsible for the typical aroma of processed foods. According to the Flavourings Regulation (EC) No. 1334/2008, heating must not exceed 15 minutes at a maximum of 180° C. for the production of reaction flavors that are used as food additives. This limitation is intended to roughly correspond to the otherwise typical kitchen conditions when processing food.

In the meat sector, the formation of reaction flavors, such as unsubstituted, mono-, or poly-substituted pyrazines, from aroma precursors as part of the Maillard reaction and the Strecker degradation of amino acids has already been extensively researched (see, for example: Belitz, H.-D., Grosch, W., Schieberle, P. (2007): Textbook of Food Chemistry. 6th ed., Springer Verlag, Heidelberg, pp. 378-382; and R. Wilhelm (2015), “Vergleichende Untersuchungen zu flüchtigen Aromastoffen beim Braten von Rind-und Schweineleber sowie von gebratener Gänse-und Gänsestopfleber” (Comparative studies on volatile aroma compounds during the roasting of beef and pork liver as well as of roasted goose liver and goose foie gras), Dissertation, Hannover University of Veterinary Medicine). Pyrazines are formed during the dimerization of α-amino ketones, which are formed during the oxidative deamination and decarboxylation of α-amino acids with α-dicarbonyl compounds. However, the use of animal material as a starting material is questionable from an animal welfare perspective and is also undesirable from a consumer perspective. Furthermore, the quantities obtained in this way are very small and therefore not economically viable.

Document DE 20 2021 104 269 U1 which describes a process for producing a formulation containing at least one (alkyl) pyrazine can be regarded as the closest prior art to the present invention. This prior art process allows to produce a natural corylone pyrazine (CP) from cyclotene and L-serine with acetic acid, without the use of metal compounds or animal starting materials. A drawback of this prior art process is that L-serine is to be used in excess: the ratio of cyclotene to L-serine is preferably 1:3. Furthermore, the prior art process uses a highly flammable solvent such as diethyl ether to isolate the desired product.

Therefore, there remains a need for a process for producing a formulation containing at least one (alkyl) pyrazine, which does not have any of the aforementioned drawbacks and which furthermore does not use metal catalysts nor material of animal origin. A further object of the invention is to proceed in the most economically viable and resource-efficient way possible.

This object was surprisingly easily achieved by a process for producing a formulation. This process comprises the following steps:
(a) providing at least one compound according to formula (II) or (III) or (IIIa), preferably a compound according to formula (II) or (III),

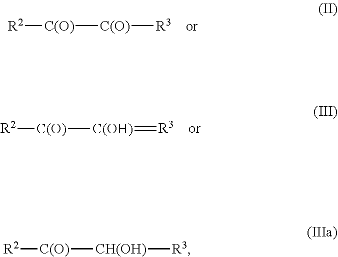

R² and R³ are identical or different and independently of one another represent hydrogen or C₁₋₄ alkyl; or
R² and R³ are bonded to one another and together form a group of the formula

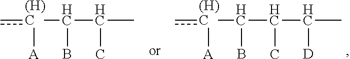

wherein
A, B, C, and D independently of one another represent hydrogen or C₁₋₄ alkyl,
(b) providing an alpha-amino acid,
(c) bringing into contact at least one compound according to formula (II) or formula (III) or formula (IIIa), preferably at least one compound of formula (II) or formula (III), and at least one alpha-amino acid from step (b);
   wherein at least one solvent suitable for the production of food products and having a boiling point of at least 140° C. is used, preferably propylene glycol, glycerol, triethyl citrate, diacetin, monoacetin, and/or triacetin, and wherein the solvent is added prior to step (c) and/or in step (c).

Thus, according to the invention, the reaction is carried out in solution using the solvent or solvent mixture described herein. It is particularly preferred to use a solvent mixture which comprises the solvent suitable for food production and having a boiling point of at least 140° C. The term “boiling point” as used herein means the boiling point at atmospheric pressure (1013.25 mbar).

It is therefore also possible to use a solvent combination in which at least one of the solvents has a boiling point of at least 140° C. The solvent having a boiling point of at least 140° C. is preferably selected from the group consisting of propylene glycol, glycerol, diacetin, monoacetin, triacetin, triethyl citrate, or mixtures thereof. Water, ethanol, and/or propanol can be added as additional solvents.

During this process, the chemical transformation shown below takes place.

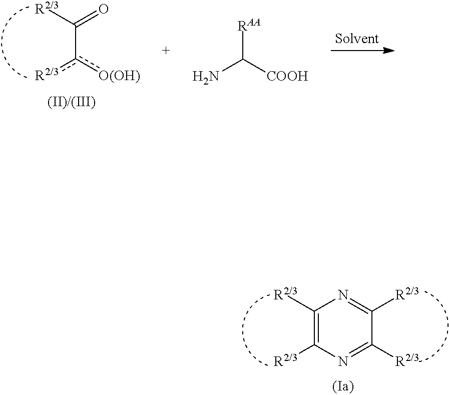

To produce an (alkyl) pyrazine of formula (Ia), any alpha-amino acid may be used, while the proteinogenic alpha-amino acids are preferred. The proteinogenic alpha-amino acid is selected from the group consisting of alanine, arginine, asparagine, aspartic acid, cysteine, glutamine, glutamic acid, glycine, histidine, isoleucine, leucine, lysine, methionine, phenylalanine, proline, serine, selenocysteine, threonine, tryptophan, tyrosine, valine, and mixtures thereof.

The process according to the invention allows to produce previously not commercially used pyrazines, such as the 2,5(6)-diethyl-3,6(5)-dimethylpyrazines obtained from acetylpropionyl (10) with an amino acid. The isolated isomer mixture exhibits a potent peanut aroma even at low doses of 10 ppm in a product. The product can be selected from the group consisting of food products, semi-luxury food products, cosmetic or pharmaceutical products, and dietary supplements.

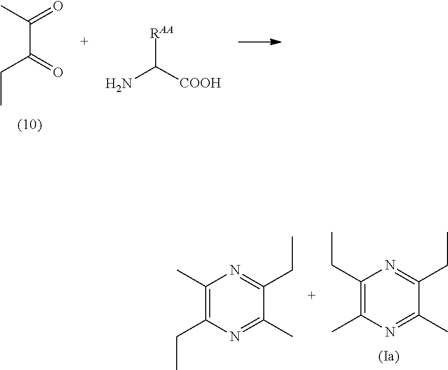

In the process according to the invention, it is particularly preferred that at least one of the solvents used comprises at least one hydroxy group. This hydroxy group carrying solvent may be selected from the group consisting of propylene glycol (PG), glycerol (also known as propane-1,2,3-triol), diacetin, monoacetin, triethyl citrate, and mixtures thereof. These hydroxy group carrying solvents may be mixed with at least one other solvent, such as triacetin or water.

If only one diketone of formula (II) or (III) is brought into contact with at least one alpha-amino acid according to the process of the invention, at least one “symmetric” (alkyl) pyrazine of formula (Ia) can be obtained in both regioisomers. For example, the following “symmetric” (alkyl) pyrazines of formula (Ia) may be obtained in the process according to the invention:

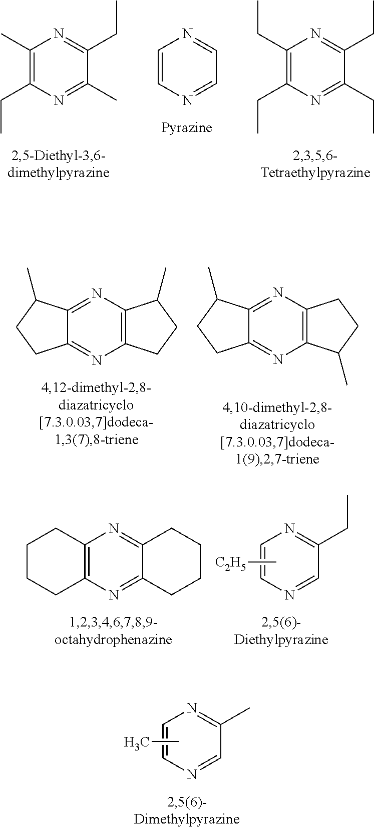

When two or more different diketones of formula (II) or (III) are brought into contact in the presence of at least one alpha-amino acid according to the process of the invention, at least one “asymmetric” (alkyl) pyrazine of formula (Ia′) can be obtained. An “asymmetric” (alkyl) pyrazine of formula (Ia′) is understood to mean a pyrazine in which the pyrazine ring is formed from two different diketones of formula (II) or (III).

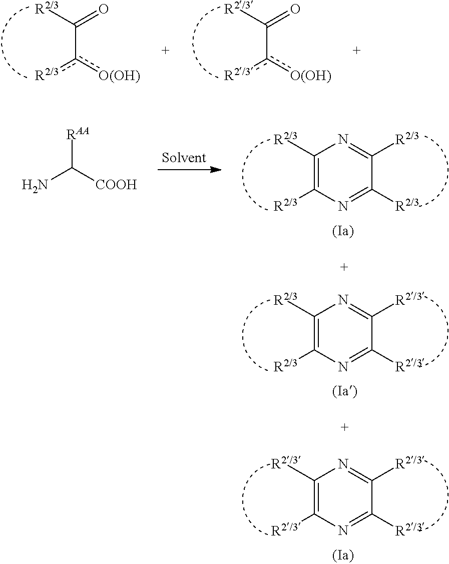

In the (alkyl) pyrazine of formula (Ia′)
R^{2′} and R^{3′} are identical or different and independently of one another represent hydrogen or C₁₋₄ alkyl; or
R^{2′} and R^{3′} are bonded to one another and together form a group of the formula

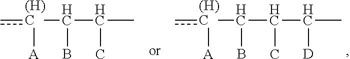

wherein A, B, C, and D independently of one another represent hydrogen or C₁₋₄ alkyl,

The process according to the invention allows to obtain, for example, the following “asymmetric” (alkyl) pyrazines of formula (Ia′):

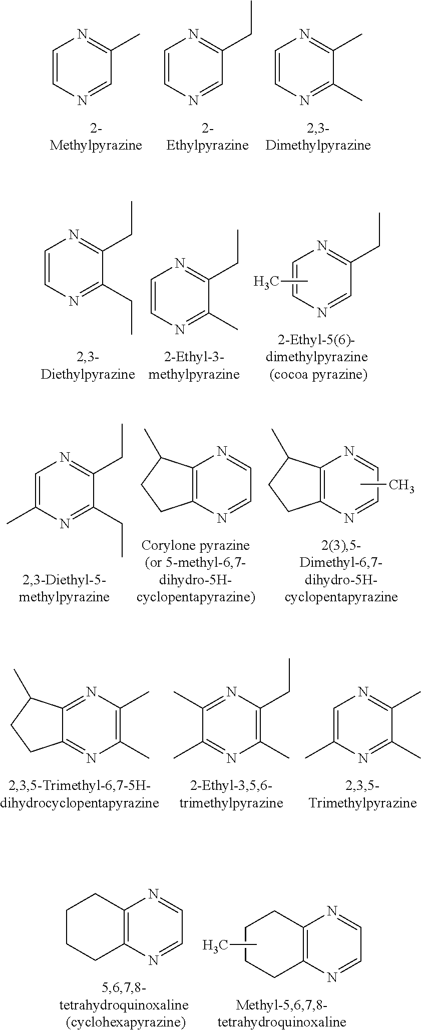

Surprisingly, it was found that when a compound of formula (II) or (III) is used in combination with at least one alpha-amino acid, preferably an alpha-amino acid of formula (IV), a second (alkyl) pyrazine of formula (Ib) is formed in addition to (Ia).

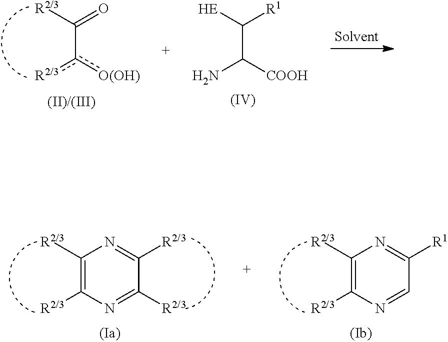

In the alpha-amino acid of formula (IV), E represents S or O, and R¹ represents hydrogen or methyl. The alpha-amino acids of formula (IV) include serine, cysteine, threonine, and mixtures thereof.

For the (alkyl) pyrazine of formula (Ib), the alpha-amino acid, preferably the alpha-amino acid of formula (IV), plays a crucial role, as its carbon skeleton is incorporated into the (alkyl) pyrazine. This means that the alpha-amino acid of formula (IV) functions not only as a nitrogen source, as described in the literature (J. AGR. FOOD CHEM., VOL. 20, NO. 5, 1972 1081), but also as a reducing agent in the Strecker degradation.

In a preferred embodiment of the invention, no organic acid such as acetic acid or propionic acid is used in the process, with the exception of the alpha-amino acid(s). In a further preferred embodiment of the invention, the formulations do not contain any organic acids such as acetic acid or propionic acid, with the exception of the alpha-amino acid(s).

Influence of the Solvent on the Ratio of the Two (Alkyl) Pyrazines (Ia) and (Ib) in the Reaction with the Amino Acid, Preferably the Amino Acid of Formula (IV):

As already mentioned above, the obtaining of (alkyl) pyrazines of formula (Ib) is highly preferred when using amino acids of formula (IV), since here the carbon skeleton of the amino acid is incorporated into the pyrazine. The process described in the present invention allows to significantly influence the ratio of the two pyrazines, such as (alkyl) pyrazine (Ia) and (alkyl) pyrazine (Ib), by selectively choosing the reaction parameters in order to obtain the desired (alkyl) pyrazine in high yield.

Surprisingly, it was found that the choice of the solvent in the process according to the invention, in particular the addition of solvents carrying hydroxy groups, preferably propylene glycol, glycerol (also known as propane-1,2,3-triol), monoacetin, triethyl citrate and/or diacetin, can impact the ratio of the two pyrazines according to formula (Ia) and (Ib) as well as their overall yield.

It was found that, when a solvent carrying one or more hydroxy group(s), preferably glycerol or propylene glycol, is used as the sole solvent, the (alkyl) pyrazine of formula (Ia) is formed particularly preferentially and in short reaction times. When the proportion of the solvent carrying one or more hydroxy group(s) is reduced (and the proportion of triacetin is increased accordingly), the percentage of the obtained (alkyl) pyrazine of formula (Ib) increases. However, since this also has a negative impact on the reaction rate, the overall yield consisting of (alkyl) pyrazines (Ia) and (Ib) may decrease if the solvent carrying the hydroxy group(s) is omitted, and additional costs can be incurred due to long reaction times. It was observed that the addition of catalytic amounts of at least one solvent carrying one or more hydroxy group(s), such as glycerol or propylene glycol, i.e. of 0.01 to 5 mol % based on the compound according to formula (II) or (III), shows the greatest yield-enhancing effect.

This solvent-dependent product selectivity now also enables cost-efficient access to new, previously not commercially exploited pyrazines, such as 2,5(6)-diethyl-3,6(5)-dimethylpyrazine obtained from acetylpropionyl (10) with an amino acid according to formula (IV). The isolated isomer mixture (i.e, the mixture of 2,5-diethyl-3,6-dimethylpyrazine and 2,6-diethyl-3,5-dimethylpyrazine) exhibits a potent peanut aroma even at low doses of 10 ppm in a product such as a food product. As shown in the table below, a higher ratio of 2,5(6)-diethyl-3,6(5)-dimethylpyrazine (Ia) to the (alkyl) pyrazine of formula (Ib) can be obtained by appropriate choice of the solvent.

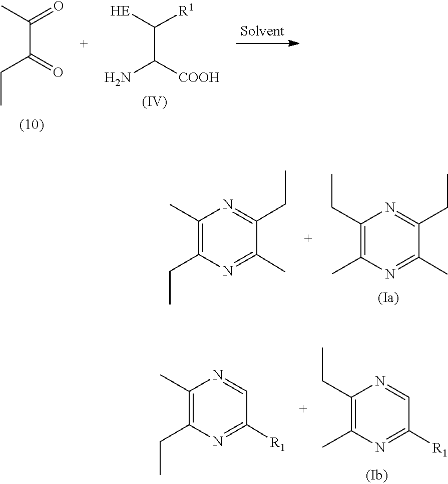

| Solvent | | Ratio (Ia):(Ib) |
|---|---|---|
| Triacetin | Glycerol or propylene glycol (ca. 0.1 mol%) | 2.3:1 |
| — | Glycerol or propylene glycol | 4.9:1 |

If a higher proportion of (alkyl) pyrazines of formula (Ib) is desired in the reaction with the alpha-amino acid of formula (IV), triacetin or a combination of solvents comprising triacetin should be used. Preferred is a mixture of triacetin and at least one solvent comprising at least one hydroxy group. This solvent carrying one or more hydroxy group(s) may be selected from the group consisting of propylene glycol, glycerol, diacetin, monoacetin, triethyl citrate, water, and mixtures thereof. The reaction time in a combination of solvents comprising triacetin is shorter than the reaction time in pure triacetin as a solvent. Particular preference is given to a mixture of triacetin and diacetin or a mixture of triacetin and glycerol. Triacetin (or glycerol triacetate, E 1518) is an ester of glycerol and acetic acid. In the food industry, it is used as a softener for chewing gum or as a flavor carrier, among other things. Triacetin is a natural ingredient of papayas.

For the yield of the (alkyl) pyrazine of formula (Ib) it is particularly advantageous if, in step (c), the reaction mixture has a ratio of triacetin to diacetin in the range from 99:1 to 1:1, preferably in the range from 20:1 to 5:1, most preferably 19:1. Surprisingly, it was found that glycerol has just as pronounced an effect as diacetin, even in smaller amounts. It is particularly advantageous if, in step (c), the reaction mixture has a ratio of triacetin to glycerol in the range from 99:1 to 99.99:0.01, preferably in the range from 99.5:0.5 to 99.9:0.1, most preferably 99.84:0.16.

The mixture obtained by the process according to the invention, which comprises (alkyl) pyrazines of formula (Ia) and (alkyl) pyrazines of formula (Ib), can be used for producing products such as food products, semi-luxury food products, cosmetic or pharmaceutical products, and dietary supplements.

The (alkyl) pyrazines of formula (Ia) and (alkyl) pyrazines of formula (Ib) can be separated from each other. For this purpose, any separation technique known to the person skilled in the art can be used. For example, distillation, crystallization or chromatography can be used as the separation techniques. The (alkyl) pyrazines of formula (Ia) or (alkyl) pyrazines of formula (Ib) isolated by these separation techniques can be used to produce products such as food products, semi-luxury food products, cosmetic or pharmaceutical products, and dietary supplements.

For example, when reacting 2,3-pentanedione with L-threonine in a solvent mixture comprising triacetin and glycerol (in catalytic amounts), at least two different pyrazine fractions were isolated, i.e. separated from each other. The first pyrazine fraction contains (alkyl) pyrazines of formula (Ib), namely cocoa pyrazine, which is composed of two isomers (2-ethyl-3,5-dimethylpyrazine and 2-ethyl-3,6-dimethylpyrazine). It was found that the isomer ratio in the cocoa pyrazine product produced by the process according to the invention differs greatly from the isomer ratio in the commercially available cocoa pyrazine product. While the commercially available product has a ratio of cocoa pyrazine isomer 1 (2-ethyl-3,5-dimethylpyrazine) to cocoa pyrazine isomer 2 (2-ethyl-3,6-dimethylpyrazine) of approximately 40:60, the ratio of cocoa pyrazine isomer 1 to cocoa pyrazine isomer 2 is about 60:40 or higher in the fraction produced by the process according to the invention. Surprisingly, it was found that the ratio of cocoa pyrazine isomers has a strong impact on the sensory potency of the overall cocoa pyrazine product. The cocoa pyrazine fraction produced by the process according to the invention is significantly more potent than the variant available on the market (such as ethyl dimethyl pyrazine from “Riverside Aromatics”), i.e. by having approximately 20% more flavor, for example. This makes the cocoa pyrazine product of the invention more economically viable (lower cost of use).

Therefore, in a preferred embodiment, the invention relates to a formulation comprising 2-ethyl-3,5-dimethylpyrazine and 2-ethyl-3,6-dimethylpyrazine in a molar ratio of at least 50:50, preferably at least 55:45, most preferably at least 60:40. Particularly preferred is a formulation comprising 2-ethyl-3,5-dimethylpyrazine and 2-ethyl-3,6-dimethylpyrazine in a molar ratio of between 50:50 and 70:30, preferably between 55:45 and 65:35. In a further preferred embodiment, the invention relates to a formulation comprising 2-ethyl-3,6-dimethylpyrazine and 2-ethyl-3,5-dimethylpyrazine in a molar ratio of at least 50:50, preferably at least 55:45, most preferably at least 60:40. Particularly preferred is a formulation comprising 2-ethyl-3,6-dimethylpyrazine and 2-ethyl-3,5-dimethylpyrazine in a molar ratio of between 50:50 and 70:30, preferably between 55:45 and 65:35. The invention also relates to a product, in particular a food product, semi-luxury food product, cosmetic or pharmaceutical product, and dietary supplement, which comprises such a formulation. Furthermore, the present invention relates to a process for producing a formulation comprising 2-ethyl-3,5-dimethylpyrazine and 2-ethyl-3,6-dimethylpyrazine, wherein the process comprises reacting 2,3-pentanedione with an alpha-amino acid, preferably L-threonine, in a solvent mixture which comprises at least one solvent comprising at least one hydroxy group, the solvent mixture preferably comprising triacetin and glycerol.

The second pyrazine fraction obtained consisted of the (alkyl) pyrazines of formula (Ia), namely the isomer mixture of 2,5-ethyl-3,6-dimethylpyrazine and 2,6-ethyl-3,5-dimethylpyrazine.

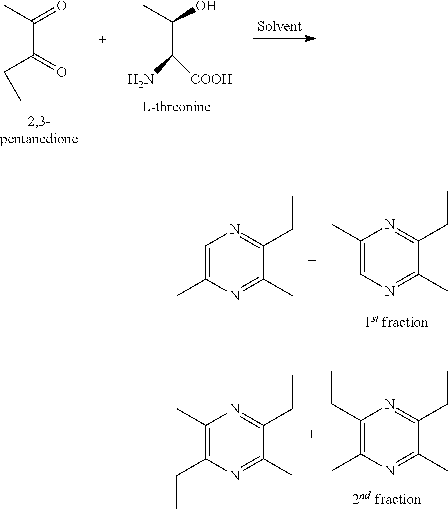

According to a further aspect, the present invention relates to a process for producing a formulation, comprising the steps of:
(a) providing at least one compound according to formula (II) or (III) or (IIIa)
   R²—C(O)—C(O)—R³ (II) or R²—C(O)—C(OH)═R³ (III) or R²—C(O)—CH(OH)—R³ (IIIa), wherein
   R² and R³ are identical or different and independently of one another represent hydrogen or C₁₋₄ alkyl; or
   R² and R³ are bonded to one another and together form a group of the formula

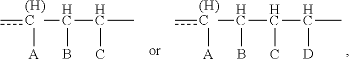

wherein
A, B, C, and D independently of one another represent hydrogen or C₁₋₄ alkyl,
(b) providing an alpha-amino acid, in particular a proteinogenic alpha-amino acid,
(c) bringing into contact at least one compound according to formula (II) or formula (III) or formula (IIIa) and at least one alpha-amino acid;
   wherein at least one solvent comprising at least one hydroxy group is added prior to step (c) and/or in step (c). Thus, according to the invention, the reaction is carried out in solution, using the solvent or solvent mixture described herein.

The solvent comprising at least one hydroxy group is preferably selected from the group consisting of propylene glycol, glycerol (also known as propane-1,2,3-triol), monoacetin, triethyl citrate, diacetin, and mixtures thereof.

It is preferred that the solvent comprising at least one hydroxy group forms part of a solvent mixture that is added prior to step (c) and/or in step (c). In a preferred embodiment, the solvent comprising at least one hydroxy group accounts for between 0.01 and 10 percent by weight of the solvent mixture. In a particularly advantageous embodiment, the solvent mixture comprises a solvent that does not comprise a hydroxy group, preferably triacetin.

In a preferred embodiment, the solvent comprising at least one hydroxy group is used in a ratio of 0.01 to 5 mol % based on the compound of formula (II) or (III) or (IIIa).

According to a further aspect, the invention relates to the use of a solvent comprising at least one hydroxy group to increase the yield in a process for producing (alkyl) pyrazine, in particular (alkyl) pyrazine of formula (Ia),

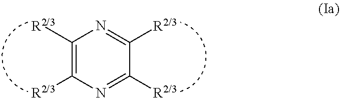

and/or (alkyl) pyrazine of formula (Ib)

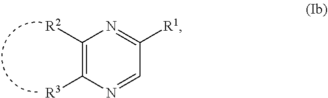

wherein
R¹ represents hydrogen or methyl,
R² and R³ are identical or different and independently of one another represent hydrogen or C₁₋₄ alkyl; or
R² and R³ are bonded to one another and together form a group of the formula

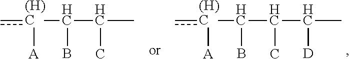

wherein A, B, C, and D independently of one another represent hydrogen or C₁₋₄ alkyl.

Particularly preferably, this process for producing (alkyl) pyrazine is the process as described above, in which at least one solvent comprising at least one hydroxy group is added prior to step (c) and/or in step (c).

According to a further aspect, the invention relates to a use of a solvent comprising at least one hydroxy group for regulating the molar ratio of the (alkyl) pyrazines of formula (Ia) and formula (Ib) in a process for producing (alkyl) pyrazines,

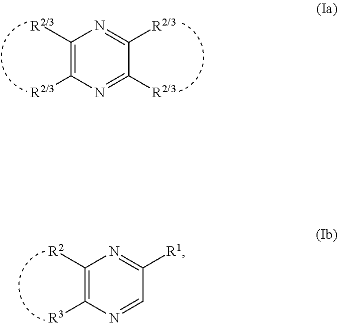

wherein
R¹ represents hydrogen or methyl,
R² and R³ are identical or different and independently of one another represent hydrogen or C₁₋₄ alkyl; or
R² and R³ are bonded to one another and together form a group of the formula

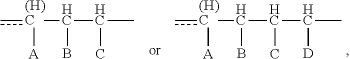

wherein A, B, C, and D independently of one another represent hydrogen or C₁₋₄ alkyl.

Particularly preferably, this process for preparing (alkyl) pyrazine is the process as described above, in which at least one solvent comprising at least one hydroxy group is added prior step (c) and/or in step (c).

According to a further aspect, the invention relates to a process for producing a formulation, comprising the following steps:
(a) providing at least one compound according to formula (II) or (III)

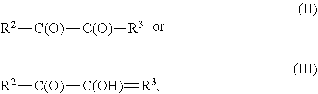

R² and R³ are identical or different and independently of one another represent hydrogen or C₁₋₄ alkyl; or
R² and R³ are bonded to one another and together form a group of the formula

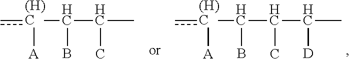

wherein
A, B, C, and D independently of one another represent hydrogen or C₁₋₄ alkyl,
(b) providing an alpha-amino acid, in particular a proteinogenic alpha-amino acid,
(c) bringing into contact at least one compound according to formula (II) or formula (III) and at least one alpha-amino acid;
   wherein the reaction is carried out in solution,
   and wherein at least one solvent suitable for the production of food products and having a boiling point above 140° C. is used.

The features of the embodiments according to the various aspects of the invention can also be combined with each other.

### Addition of Ammonium Salt

Due to the relatively high cost of natural quality amino acids, in particular L-serine, which was used as the sole nitrogen source in the process according to claim 1, a more cost-effective alternative was sought.

This object was surprisingly easily achieved using a process according to claim 2. The process comprises additionally adding at least one ammonium salt. It is particularly advantageous if at least one ammonium salt is added in a single dose in step (c).

It was found that by using an ammonium salt, the amount of an alpha-amino acid, preferably an alpha-amino acid of formula (IV), can be reduced by 50%, while the yield of the target product, i.e. (alkyl) pyrazine of formula (Ib), remains the same. If the amount of amino acid is not reduced, i.e, the molar ratio of the compound of formula (II) or (III) or (IIIa) to the alpha-amino acid remains 1:2, a significant increase in yield is achieved when adding an ammonium salt, preferably 2 equivalents of the ammonium salt. Furthermore, it can also be observed that fewer by-products are formed during purification, which considerably simplifies the process.

The ammonium salt used is preferably an inorganic or organic ammonium salt. Particularly preferably, an ammonium salt is selected from the group consisting of ammonium halide, ammonium phosphates, ammonium sulfates, ammonium carbonate, ammonium hydrogen carbonate, and ammonium carbamate, the ammonium salt of an organic acid, and mixtures thereof. Ammonium chloride is particularly preferred.

Here, ammonium salt is added in a molar ratio to the amino acid, preferably to the amino acid of formula (IV), the ratio being in a range from 4:1 to 0.1:1, preferably in the range from 2:1 to 0.5:1, most preferably a ratio of 1:1.

The solvent, preferably at least one organic solvent, most preferably triacetin, may be added prior to step (c) and/or in step (c). More particularly, the solvent may be added prior to step (c) to the compound according to formula (II) or (III) or (IIIa) and/or to an alpha-amino acid, preferably to an alpha-amino acid of formula (IV). In an advantageous embodiment, the process according to the invention comprises, prior to step (c), a further step
(c1) of adding at least one solvent to the compound according to formula (II) or (III) or (IIIa), whereby a solution is obtained in which the amount of the compound according to formula (II) or (III) or (IIIa) depends on the solubility of this or these compound(s) in the solvent or solvent combination used. A solution saturated to a maximum with the compound of formula (II) or (III) or (IIIa) is preferred. Most preferably, the amount of the compound of formula (II) or (III) or (IIIa) in the solvent is up to 40 wt %, most preferably about 2 to 10 wt %, based on the total weight of this solution.

In step (c), the solution obtained in step (c1) is added to the alpha-amino acid either dropwise or in one dose, with constant stirring. The mixture is heated to a maximum temperature of about 180° C., preferably about 100 to 140° C., most preferably 110 to 130° C.

It was found to be particularly advantageous if, prior to step (c), the solution obtained in step (c1) is divided into two doses, the ratio of the first dose to the second dose being in the range from 1:10 to 1:4, preferably in the range from 1:7 to 1:3, most preferably 1:4.

In a further advantageous embodiment, the process according to the invention comprises, following step (c1), a further step
(c2) of mixing the first dose of the solution obtained in step (c1) with the alpha-amino acid, preferably with the alpha-amino acid of formula (IV), and heating this mixture to a maximum temperature of about 180° C., preferably about 100 to 140° C., most preferably 110 to 130° C.

In step (c), the second dose of the solution obtained in step (c1) is added dropwise to the mixture obtained in step (c2), with constant stirring.

In a further advantageous embodiment of the invention, water, especially distilled water, is used as a solvent in addition to triacetin. It was found that the amount of water can have an impact on the yield. It is particularly advantageous if, in step (c), the reaction mixture has a ratio of water to the compound according to formula (II) or (III) or (IIIa) in the range from 40:1 to 10:1, preferably in the range from 30:1 to 20:1, most preferably 21:1.

The process according to the invention may additionally comprise the addition of water in step (c). The water may be added in one dose to the mixture comprising the alpha-amino acid and the solution obtained in step (c1) which comprises at least one compound of formula (II) or (III) or (IIIa).

Furthermore, it was found that particular advantages in terms of increasing the yield and shortening the reaction time are achieved by adding a solvent mixture, such as triacetin and diacetin, in combination with the adding of at least one ammonium salt.

Moreover, it was found that the enhanced reaction control with the addition of an ammonium salt also allows for a reduction in the amount of solvent or solvent mixture. The total reaction volume can be halved without a significant loss in yield (less than 4 mol %), which was not possible without the addition of an ammonium salt. This not only increases the economic efficiency of the process, but also leads to simplified purification, a comparably higher yield and, last but not least, a more environmentally friendly alternative with half the amount of waste.

In an alternative embodiment of the invention, step (c) comprises adding water to the mixture obtained in step (c2) in a dropwise manner, with constant stirring. The water is added simultaneously with the adding of the solution obtained in step (c1). However, the water is added separately, i.e. without mixing it with the second dose of the solution obtained in step (c1). The rate of adding water is comparable to the rate of adding the solution obtained in step (c1).

In the context of the present invention, the radical “C₁₋₄ alkyl” in the compound of formula (II) or (III) or (IIIa) stands for methyl, ethyl, propyl, or butyl, preferably methyl.

The compound of formula (II) or (III) or (IIIa) is preferably selected from the group consisting of

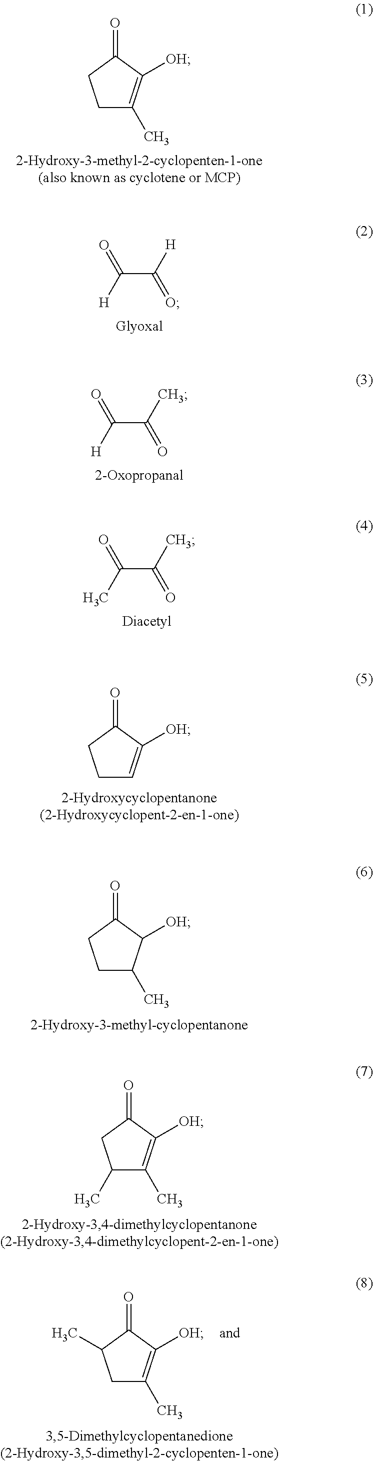

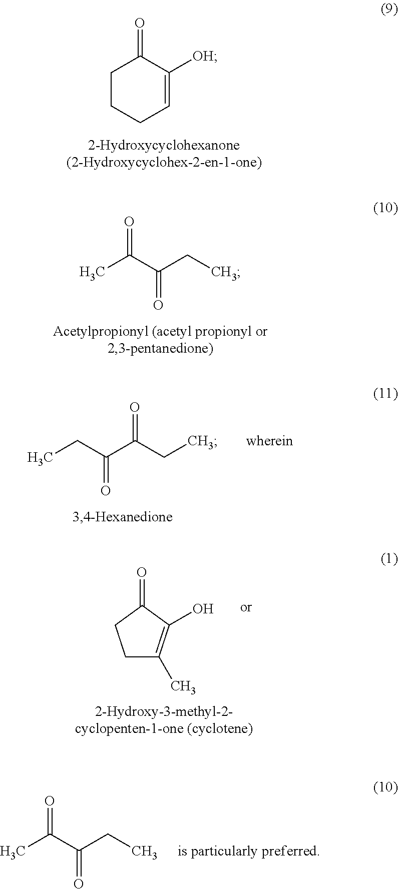

The alpha-amino acid of formula (IV) is in particular selected from the group consisting of L-/D-/(RS)-serine, L-/D-/(RS)-cysteine, L-/D-/(RS)-threonine, and mixtures thereof, preferably L-/D-/(RS)-serine and L-/D-/(RS)-cysteine, particularly preferably L-serine or L-cysteine, most preferably L-serine. The amino acids used may be obtained, for example, by enzymatic hydrolysis of vegetable protein, if the aromatizing end products or final substances obtained from these amino acids are desired in a natural quality.

The molar ratio of a compound of formula (II) or (III) or (IIIa) to the alpha-amino acid, preferably to the alpha-amino acid of formula (IV), may be in the range from 1:1 to 1:5. In the process known from DE 202021104269 U1, the preferred ratio of a compound of formula (II) or (III) or (IIIa) to the alpha-amino acid of formula (IV) is 1:3. Surprisingly, it was found that in the process according to the invention, this ratio can be as low as 1:2, for example between 1:1 and 1:2. This results in economic advantages of the process according to the invention.

The reaction in step (c) is accomplished at maximum temperatures of approximately 180° C., preferably at temperatures in the range from approximately 100 to 140° C., more preferably in the range from approximately 110 to 130° C., and most preferably 125° C. In the course of the reaction, water was observed to be formed. The reaction in step (c) takes place in particular over a duration of up to 40 hours, preferably over a duration of up to 24 hours, more preferably from 1 to 12 hours, most preferably up to 6 hours.

The progress of the reaction in step (c) can be monitored by gas chromatography (GC) analysis. When no more compound of formula (II) or (III) is detected, the reaction is stopped, for example by cooling the reaction mixture to room temperature and, optionally, the reaction mixture is neutralized using a basic solution. Preferably, a solution of alkali hydroxides, such as sodium or potassium hydroxide solution, may be used as the basic solution.

Following step (c), the process may additionally comprise a further step
(c1) of isolating the resulting (alkyl) pyrazine of formula (Ia) and/or (Ib) from the crude product obtained from step (c).

The isolating in step (c1) is preferably carried out by rectification, optionally under reduced pressure. Alternatively, after adjusting the pH, the crude product may be concentrated and purified by solid phase extraction (SPE). The fraction(s) comprising (alkyl) pyrazine of formula (Ia) and/or (Ib) can be obtained with a purity of at least 95 wt %. The fractions obtained by the process according to the invention may comprise one or more (alkyl) pyrazines. The fraction comprising a plurality of (alkyl) pyrazines may optionally be further used as formulation(s). For example, a fraction may be isolated which comprises both trimethylpyrazine and tetramethylpyrazine.

Another extraction of the crude product from the reaction mixture may be accomplished by co-distillation with propylene glycol (PG). To this end, a mixture of glycerol and PG (3:2) is added to the reaction mixture at room temperature and is subsequently distilled under reduced pressure. The PG distilled in this way serves as a carrier agent for the pyrazine formed.

In a particularly preferred embodiment of the process, triacetin or the triacetin-comprising mixtures is/are added as a solvent, while using 2-hydroxy-3-methyl-2-cyclopenten-1-one (cyclotene) as the compound of formula (II) or (III) and L-serine as the alpha-amino acid of formula (IV). Not only is triacetin able to dissolve cyclotene, it may also offer enormous advantages in the isolation of the desired product, such as corylone pyrazine. Triacetin has a higher boiling point than corylone pyrazine, so that corylone pyrazine, as the target product, can be isolated to a desired purity by rectification.

Further improvement of the process in terms of yield, selectivity and atom economy has been observed when adding a solvent mixture comprising triacetin and diacetin in combination with the addition of ammonium chloride.

The closest prior art, document DE 20 2021 104 269 U1, discloses the isolation of the desired product, such as corylone pyrazine, by extraction in a Likens-Nickerson apparatus with diethyl ether. Diethyl ether forms highly flammable vapor-air mixtures, making its use undesirable in the industry. The use of triacetin as a solvent allows to avoid using diethyl ether.

Moreover, surprisingly, an increase in yield of the target product was observed in comparison to the process known from DE 20 2021 104 269 U1. The yield of corylone pyrazine may be up to approximately 30 mol % in the present inventive process, whereas in the process described in DE 20 2021 104 269 U1, by contrast, the yield of corylone pyrazine is only approximately 6 mol %. The corylone pyrazine-comprising fraction obtained after purification and isolation may have a purity of at least 95 wt %.

Further subject-matter encompassed by the present invention includes the formulation which is in particular produced by the process according to the invention and which comprises at least one (alkyl) pyrazine of formula (Ia)

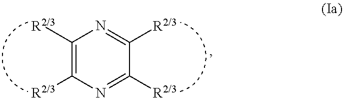

wherein
R² and R³ are identical or different and independently of one another represent hydrogen or C₁₋₄ alkyl; or
R² and R³ are bonded to one another and together form a group of the formula

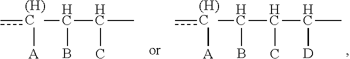

wherein A, B, C, and D independently of one another represent hydrogen or C₁₋₄ alkyl.

The formulation according to the invention may additionally comprise at least one (alkyl) pyrazine of formula (Ib)

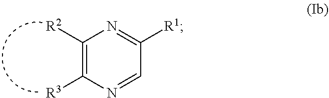

wherein
R¹ represents hydrogen or methyl,
R² and R³ are identical or different and independently of one another represent hydrogen or C₁₋₄ alkyl; or
R² and R³ are bonded to one another and together form a group of the formula

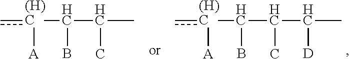

wherein A, B, C, and D independently of one another represent hydrogen or C₁₋₄ alkyl.

The (alkyl) pyrazines of formula (Ia) and the (alkyl) pyrazines of formula (Ib) can be separated from one another. For this purpose, any separation technique known to the person skilled in the art may be used. For example, distillation, crystallization, or chromatography may be used as a separation technique. The (alkyl) pyrazines of formula (Ia) or (alkyl) pyrazines of formula (Ib) isolated by these separation techniques may be used for producing products such as food products, semi-luxury food products, cosmetic or pharmaceutical products, and dietary supplements.

This (alkyl) pyrazine of formula (Ib) may be produced from at least one alpha-amino acid as the sole nitrogen source. Preferably, a combination comprising at least one alpha-amino acid and at least one ammonium salt is used as the sole nitrogen source for producing at least one (alkyl) pyrazine according to formula (Ib). Most preferably, a combination consisting of at least one alpha-amino acid and at least one ammonium salt is used as the sole nitrogen source for producing at least one (alkyl) pyrazine of formula (Ib).

The formulation preferably contains at least about 99.9 wt %, preferably at least about 95 wt %, of at least one (alkyl) pyrazine according to formula (Ib). Furthermore, the minimum amount of at least one (alkyl) pyrazine of formula (Ib) in the formulation according to the invention is about 0.01 wt %. Preferably, the formulation according to the invention may contain at least about 0.1 wt % of at least one (alkyl) pyrazine of formula (Ib) in one or more solvents suitable for the production of a food product. For example, in the case of an isolation of the formulation by SPE, in which ethanol is used as the solvent, a product can be obtained which contains at least about 0.1 to about 0.2 wt % of at least one (alkyl) pyrazine according to formula (Ib). In the case of co-distillation with PG, a product can be obtained which contains at least about 2 to about 3 wt % of at least one (alkyl) pyrazine according to formula (Ib).

Particularly preferred are the (alkyl) pyrazines of formula (Ib) in which
R¹ represents hydrogen and
R² and R³ are bonded to one another and together form a group of the formula

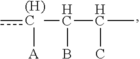

wherein
A represents methyl, and B as well as C represent hydrogen.

Most preferred are the (alkyl) pyrazines of formula (Ia) or (Ib) which are selected from the group consisting of

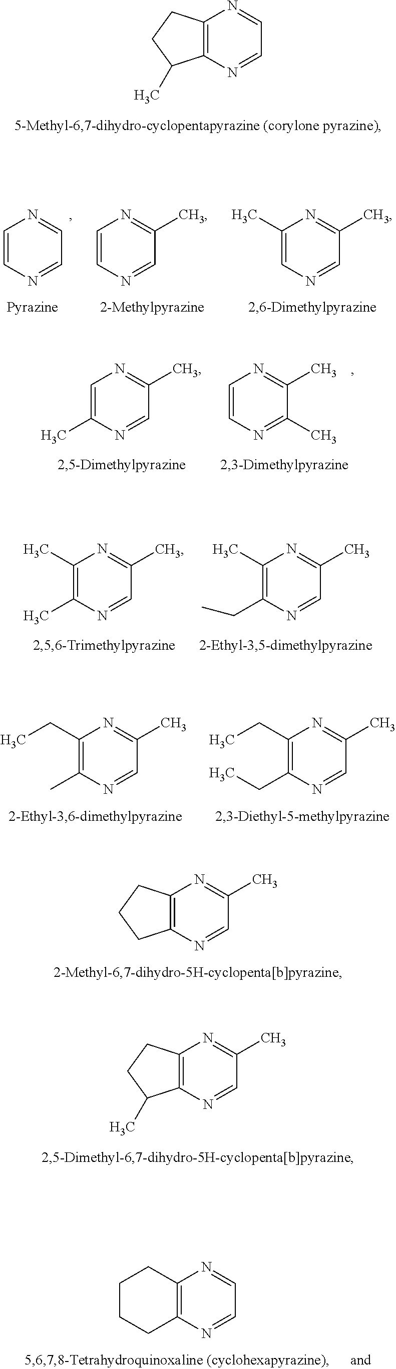

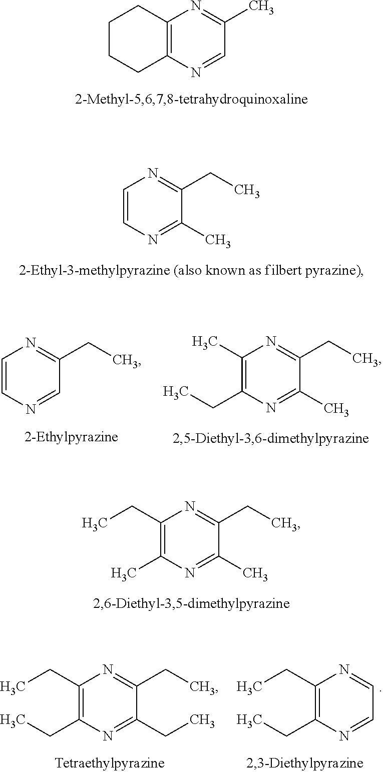

The formulation according to the invention is preferably free of alcohol, particularly preferably free of ethanol.

A formulation for aromatizing a product, such as a food product, semi-luxury food product, cosmetic or pharmaceutical product, and dietary supplement, which contains at least 95 wt % of 5-methyl-6,7-dihydro-cyclopentapyrazine (corylone pyrazine), represents preferred subject-matter of the invention.

A formulation for aromatizing a product, such as a food product, semi-luxury food product, cosmetic or pharmaceutical product, and dietary supplement, which contains at least 95 wt % of 2,5(6)-diethyl-3,6(5)-dimethylpyrazine, represents preferred subject-matter of the invention.

Further subject-matter encompassed by the present invention includes the product, in particular the food product, semi-luxury food product, cosmetic or pharmaceutical product, and dietary supplement, which comprises a formulation according to the invention. The proportion of the formulation according to the invention in this product amounts to up to approximately 1000 ppm, preferably from about 10-3 ppm to about 750 ppm, most preferably from about 0.1 to 150 ppm.

The formulation according to the invention can be used for aromatizing a product, in particular a food product, semi-luxury food product, cosmetic or pharmaceutical product, or dietary supplement. For this purpose, the product, in particular a food product, semi-luxury food product, cosmetic or pharmaceutical product, or dietary supplement, is brought into contact with the formulation according to the invention.

Furthermore, the present invention relates to a process for producing a product, in particular a food product, semi-luxury food product, cosmetic or pharmaceutical product, and dietary supplement, in which the formulation according to the invention is brought into contact with the product.

The invention shall be explained by way of the included examples, but is not limited to the embodiments specifically described. The invention also relates to all combinations of preferred implementations, insofar as these are not mutually exclusive. The terms “about” or “approximately” in combination with a numerical value are understood to mean that at least values 10% higher or lower, or 5% higher or lower, and in any case 1% higher or lower, shall be included.

### EXAMPLE 1

### Starting Materials:

L-serine (500 mmol, 52.5 g)
cyclotene (250 mmol, 28 g) as a 5% solution in triacetin (560 g)
distilled water (95 g)

L-serine (500 mmol, 52.5 g) is mixed with 112 g of cyclotene solution (5% solution in triacetin), and the mixture is heated to approximately 110° C.

The remaining cyclotene solution (448 g) and the distilled water are each provided in a dropping funnel and only added when the temperature of the mixture reaches 110° C. The mixture is stirred at 110 to 120° C. Progress of the reaction is monitored by GC analysis. When cyclotene is no longer detectable in the reaction mixture, the reaction is stopped (e.g. by cooling to room temperature), and the mixture is processed using a rectification apparatus, to obtain corylone pyrazine with a purity of at least 95 wt %. The obtained product was analyzed by GC-FID.

### EXAMPLE 2

L-serine (105 mmol, 11.1 g) is mixed with 116 g of cyclotene solution (5% solution in triacetin) and with 20 g of distilled water. The mixture is heated to 120° C. with constant stirring. Progress of the reaction is monitored by GC analysis. When cyclotene is no longer detectable in the reaction mixture, the reaction is stopped, and the mixture is processed using a rectification apparatus, to obtain corylone pyrazine with a purity of at least 95 wt %.

### EXAMPLE 3

L-serine (2500 mmol, 263 g) is mixed with 600 g of cyclotene solution (5% solution in triacetin) and heated to approximately 110 to 120° C. A further cyclotene solution (2280 g) and distilled water are each provided in dropping funnels and only added when the temperature of the mixture reaches 110° C. The mixture is stirred at 110 to 120° C. Progress of the reaction is monitored by GC analysis. Once no cyclotene is detectable in the mixture, the reaction is stopped, and the mixture is adjusted to pH 12 using 30% NaOH solution. The resulting mixture is passed through an SPE column and subsequently eluted with ethanol. This ethanolic extract contains 10,000 to 12,000 ppm of corylone pyrazine and can be declared as a natural flavor extract.

### EXAMPLE 4

L-serine (121 mmol, 13.9 g) is mixed with cyclotene (121 mmol, 13.9 g), 138 g of glycerol, 91.8 g of propylene glycol, and 45.9 g of water. The mixture is heated to 120° C. Progress of the reaction is monitored by GC analysis. Once no cyclotene is detectable in the mixture, the reaction is stopped. The mixture is distilled under reduced pressure (40 mbar) by slowly heating the mixture to 160° C. and reducing the pressure to 10 mbar. The collected fraction contains 1000 to 5000 ppm of corylone pyrazine in PG.

### EXAMPLE 5

L-serine (420 mmol, 44.2 g) is provided together with cyclotene (210 mmol, 24.3 g) in a triacetin/diacetin mixture with a triacetin to diacetin ratio of 9:1 (1 L). The mixture is heated to 125° C. with constant stirring. Progress of the reaction is monitored by GC analysis. When cyclotene is no longer detectable in the reaction mixture and approximately 19 mol % of corylone pyrazine has been obtained, which will be the case after approximately 72 h, the reaction is stopped, and the mixture is processed using a rectification apparatus in order to obtain corylone pyrazine with a purity of at least 95 wt %.

### EXAMPLE 6

### Starting Materials:

L-serine (420 mmol, 44.1 g);
cyclotene (210 mmol, 24.3 g);
ammonium chloride (420 mmol, 22.5 g);
triacetin (463 g) and diacetin (24.0 g).

L-serine (2 eq.), ammonium chloride (2 eq.), and cyclotene (1 eq.) are provided together with triacetin (10 eq.) and diacetin (0.6 eq.) in a 1-liter, 3-neck flask equipped with a reflux condenser and a KPG stir bar. The mixture is heated to 125° C. with constant stirring. Progress of the reaction is monitored by GC analysis. When cyclotene is no longer detectable in the reaction mixture (after approximately 32 hours), the reaction is stopped. The yield was determined by GC analysis and is approximately 29.3 mol %, based on the molar amount of cyclotene used. The crude mixture was processed using a rectification apparatus.

### EXAMPLES 7-11

Example 7 was implemented similarly to Example 5, except that the ratio of triacetin to diacetin was 19:1 in Example 7, other than in Example 5.

Examples 8 through 12 were implemented similarly to Example 6, while the components, such as L-serine, ammonium chloride and the solvent, and their respective amounts were varied in comparison to Example 6 (see Table 1).

TABLE 1

| | | | Eq. | | Yield |
|---|---|---|---|---|---|
| Ex. | | Eq. | ammonium | Reaction | (mol %, acc. |
| # | Solvent | L-serine | chloride | time | to GC-MS) |
| 7 | Triacetin + 5% Diacetin | 2 | 0 | 72 h | 19.1% |
| 8 | Triacetin | 2 | 0 | 96 h | 16.5% |
| 9 | Triacetin | 2 | 2 | 96 h | 30.1% |
| 10 | Triacetin + 5% Diacetin | 1 | 1 | 72 h | 14.9% |
| 11 | Triacetin + 5% Diacetin | 1.5 | 1.5 | 56 h | 24.5% |
| 6 | Triacetin + 5% Diacetin | 2 | 2 | 32 h | 29.3% |
| 12 | Triacetin + 5% Diacetin, | 2 | 2 | 64 h | 27.1% |
| | half volume | | | | |

As can be seen from Table 1, the addition of ammonium chloride leads to an increase in yield. By using ammonium chloride in combination with a solvent mixture, such as triacetin and diacetin (see Example 10), the amount of L-serine can be reduced by 50%, while a comparable yield of the target product, i.e. corylone pyrazine, is achieved within a shorter reaction time compared to the reaction without ammonium chloride and without diacetin (see Example 8).

If the amount of L-serine is not reduced, a significant increase in yield and further reduction in the reaction time is observed when adding an ammonium salt in combination with a solvent mixture such as triacetin and diacetin (see example 6). Furthermore, it can also be observed that fewer byproducts interfering with the purification are produced, which considerably simplifies the process.

Moreover, it was found that the amount of solvent or solvent mixture can be reduced (see Example 12). The total reaction volume can be halved without significant loss of yield, which was not possible without the addition of an ammonium salt. This not only increases the economic efficiency of the process, but also leads to simplified purification, comparably higher yields and, last but not least, a more environmentally friendly alternative with half the amount of waste.

### EXAMPLE 12

### Starting Materials:

L-threonine (22 mol, 2.62 kg);
2,3-pentadione (11 mol, 1.1 kg);
ammonium chloride (22 mol, 1.12 kg);
triacetin (55 mol, 12.1 kg) and glycerol (8.8 mmol 0.8 g).

L-Threonine (2 eq.), ammonium chloride (2 eq.), and 2,3-pentadione (1 eq.) are provided together with triacetin (5 eq.) and glycerol (0.0008 eq.) in a 20-liter, 3-neck flask equipped with a reflux condenser and a KPG stir bar. The mixture is heated to 140° C. with constant stirring. Progress of the reaction is monitored by GC analysis. When 2,3-pentadione is no longer detected in the reaction mixture (after approximately 8 hours), the reaction is stopped. The yield was determined by GC analysis and amounted to approximately 40 mol % for the isomer mixture of 2-ethyl-3,5(6)-dimethylpyrazine ((alkyl) pyrazine of formula (Ib)) and 20 mol % for the isomer mixture of 2,5-diethyl-3,6(5)-dimethylpyrazine ((alkyl) pyrazine of formula (Ia)), based on the molar amount of 2,3-pentadione used. The crude mixture was further processed using a rectification apparatus.

### EXAMPLES 13-17

The reactions described in Examples 13-17 are reactions that were carried out in 20 mL crimp vials. Here, the intension was to investigate the different influence of the solvents and the ammonium salt on the ratio of pyrazines of formula (Ia) and (Ib). For this purpose, 0.5 g of 2,3-pentadione (2 mmol) and 1.0 g of serine (10 mmol) were dissolved in 5 mL of the respective solvents. Subsequently, the mixture was heated to 140° C. for 24 h with stirring. After cooling, the ratios of pyrazine Ia and Ib were determined by GC-FID.

TABLE 2

| | | | 2-Ethyl-3- | 2,5(6)-Diethyl- | | Ratio |
|---|---|---|---|---|---|---|
| | | | methyl- | 3,6(5)-dimethyl- | Total | pyrazine |
| | | | pyrazine | pyrazine | pyrazine | (Ia)/(Ib) |
| Ex. | | Eq. | (in mol %) | (in mol %) | yield | (acc. to |
| # | Solvent | NH₄Cl | (Ib) | (Ia) | (in mol %) | GC-FID) |
| 13 | Triac. | 2 | 20.4 | 40.1 | 60.5 | 2.0 |
| 14 | Triac. + gly. | 2 | 20.3 | 46.4 | 66.7 | 2.3 |
| 15 | Triac. + gly. | 1 | 20.0 | 41.1 | 61.1 | 2.0 |
| 16 | Triac. + gly. | 0 | 9.5 | 16.1 | 25.6 | 1.7 |
| 17 | Propylene | 2 | 9.4 | 45.8 | 55.2 | 4.9 |
| | glycol | | | | | |

As can be seen from Table 2, the adding of ammonium chloride leads to an increase in the total yield of pyrazines. This is particularly evident from Example 16, where the total yield of only 25.6% is significantly lower than the total yields of between 55.2 and 66.7% obtained in Examples 13-15 and 17.

As can be seen from the comparison of Example 13 with Examples 14-17, the addition of hydroxy groups carrying solvents such as glycerol and propylene glycol can increase the ratio of obtained pyrazine according to formula (Ia). In catalytic amounts (Examples 14 & 15, glycerol 0.15%), this increases the total yield, whereas when used as the sole solvent (Example 17), selectivity is strongly shifted toward pyrazine of formula (Ia).

1-26. (canceled)

27. A process for producing a formulation which comprises at least one (alkyl) pyrazine, comprising the steps of:
(a) providing at least one compound according to formula (II) or (III) or (IIIa) 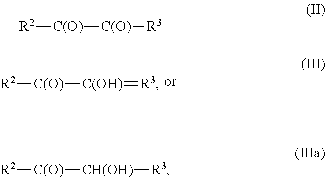
wherein
R² and R³ are identical or different and independently of one another represent hydrogen or C₁₋₄ alkyl; or
R² and R³ are bonded to one another and together form a group of the formula 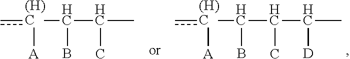
wherein
A, B, C, and D independently of one another represent hydrogen or C₁₋₄ alkyl,
(b) providing an alpha-amino acid,
(c) bringing into contact the at least one compound according to formula (II) or formula (III) or formula (IIIa) and the alpha-amino acid;
wherein at least one solvent suitable for the production of food products and having a boiling point of at least 140° C. is used;
and wherein the solvent is added prior to step (c) and/or in step (c), wherein the compound according to formula (II) or (III) or (IIIa) is selected from the group consisting of:

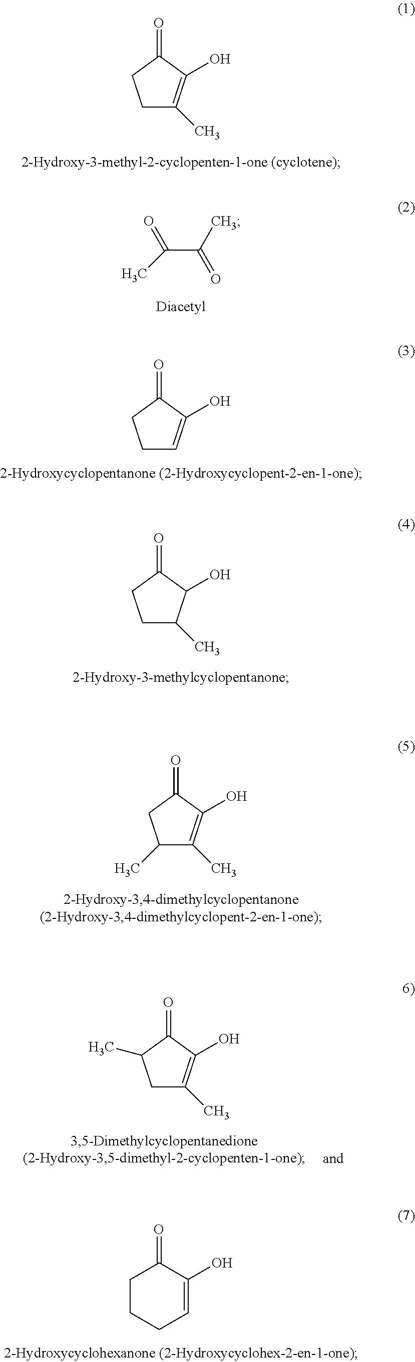

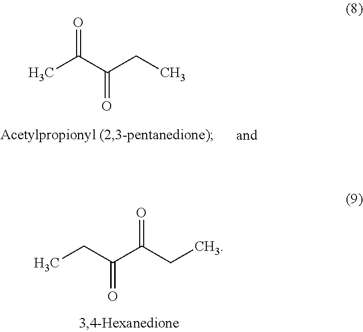

28. The process according to claim 27, wherein the process is carried out in the presence of at least one ammonium salt.

29. The process according to claim 27, wherein the alpha-amino acid in step (b) is an alpha-amino acid according to formula (IV), 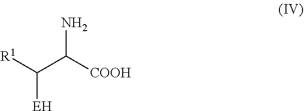
wherein E represents O or S and R¹ represents hydrogen or methyl.

30. The process according to claim 27, wherein the alpha-amino acid in step (b) is selected from the group consisting of L-/D-/(RS)-serine, L-/D-/(RS)-cysteine, L-/D-/(RS)-threonine and mixtures thereof.

31. The process according claim 27, wherein the molar ratio of a compound according to formula (II) or (III) or (IIIa) to the alpha-amino acid is between 1:1 and 1:5, and/or wherein the molar ratio of ammonium salt to the alpha amino acid is in a range from 4:1 to 0.1:1.

32. The process according to claim 27, wherein the solvent is selected from the group consisting of propylene glycol, glycerol, diacetin, monoacetin, triacetin, triethyl citrate, and mixtures thereof, wherein water, ethanol, propanol can additionally be added the solvent.

33. The process according to claim 27, wherein the reaction in step (c) is accomplished at a maximum temperature of approximately 180° C.

34. The process according to claim 27, wherein the formulation obtained comprises at least one (alkyl) pyrazine according to formula (Ia) 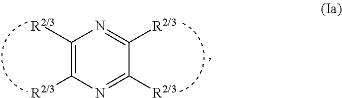
wherein
R² and R³ are identical or different and independently of one another represent hydrogen or C₁₋₄ alkyl; or
R² and R³ are bonded to one another and together form a group of the formula 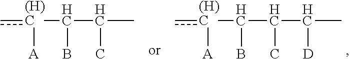
wherein A, B, C, and D independently of one another represent hydrogen or C₁₋₄ alkyl.

35-38. (canceled)

39. A process for producing a formulation which comprises at least one (alkyl) pyrazine, comprising the steps of:
(a) providing at least one compound according to formula (II) or (III) or (IIIa) 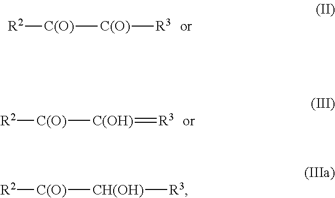
wherein
R² and R³ are identical or different and independently of one another represent hydrogen or C₁₋₄ alkyl; or
R² and R³ are bonded to one another and together form a group of the formula 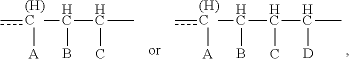
wherein
A, B, C, and D independently of one another represent hydrogen or C₁₋₄ alkyl,
(b) providing an alpha-amino acid,
(c) bringing into contact the at least one compound according to formula (II) or formula (III) or formula (IIIa) and the alpha-amino acid;
wherein at least one solvent comprising at least one hydroxy group is added prior to step (c) and/or in step (c), wherein the compound according to formula (II) or (III) or (IIIa) is selected from the group consisting of:

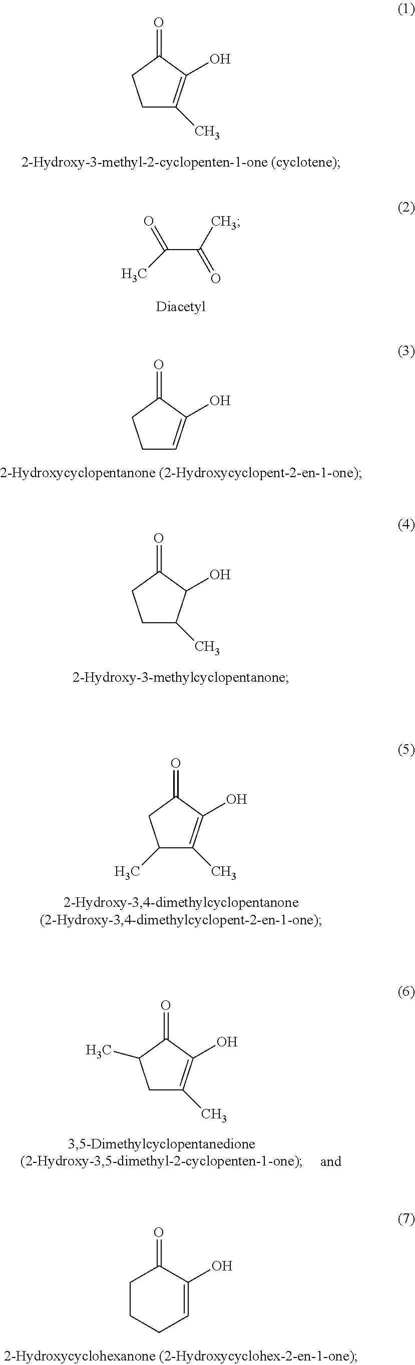

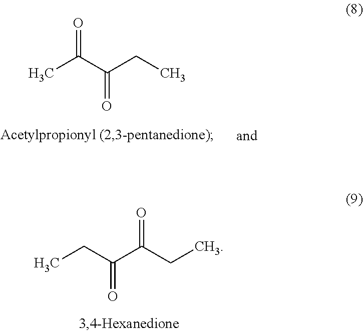

40. The process according to claim 39, wherein the solvent comprising at least one hydroxy group is propylene glycol, glycerol, monoacetin, triethyl citrate, and/or diacetin.

41. The process according to claim 39, wherein the solvent comprising at least one hydroxy group forms part of a solvent mixture which is added prior to step (c) and/or in step (c).

42. The process according to claim 41, wherein the solvent
comprising at least one hydroxy group is present in an amount of 0.01 and 10% by weight of the solvent mixture,
and/or
wherein the solvent mixture comprises a solvent which does not comprise a hydroxy group.

43. The process according to claim 39, wherein the solvent
comprising at least one hydroxy group in a ratio of 0.01-5 mol % based on the amount of the compound according to formula (II) or (III) or (IIIa) that is present.

44. A method of increasing yield in a process for producing an (alkyl) pyrazine comprising the step of carrying out a reaction for producing the (alkyl) pyrazine in a solvent comprising at least one hydroxy group,
wherein the solvent comprising at least one hydroxy group is part of a solvent mixture comprising triacetin.

45. A method for regulating molar ratios of (alkyl) pyrazines in a process for producing (alkyl) pyrazines, comprising carrying out said process in a solvent comprising at least one hydroxy group,
wherein the (alkyl) pyrazines have the structures of formula (Ia) and formula (Ib), 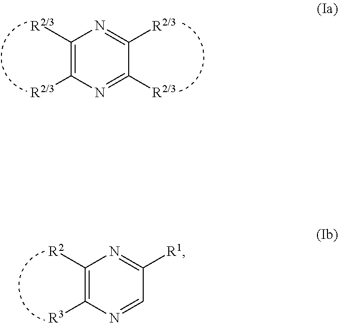
wherein
R¹ represents hydrogen or methyl,
R² and R³ are identical or different and independently of one another represent hydrogen or C₁₋₄ alkyl; or
R² and R³ are bonded to one another and together form a group of the formula 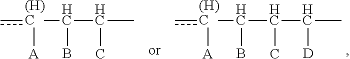
wherein A, B, C, and D independently of one another represent hydrogen or C₁₋₄ alkyl, and
wherein the solvent comprising at least one hydroxy group is part of a solvent mixture comprising triacetin.

46. The process of claim 34, wherein the formulation further comprises at least one (alkyl) pyrazine according to formula (Ib) 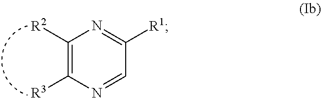
wherein
R¹ represents hydrogen or methyl,
R² and R³ are identical or different and independently of one another represent hydrogen or C₁₋₄ alkyl; or
R² and R³ are bonded to one another and together form a group of the formula 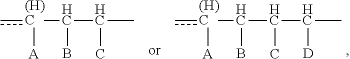
wherein A, B, C, and D independently of one another represent hydrogen or C₁₋₄ alkyl.

47. The process according to claim 33, wherein the reaction in step (c) is accomplished over a duration of up to 40 hours.

48. A formulation comprising at least one (alkyl) pyrazine according to formula (Ia), 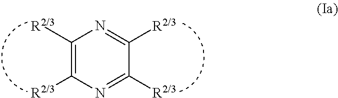
and at least one (alkyl) pyrazine according to formula (Ib) 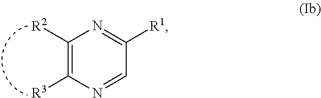
wherein
R¹ represents hydrogen or methyl,
R² and R³ are identical or different and independently of one another represent hydrogen or C₁₋₄ alkyl; or
R² and R³ are bonded to one another and together form a group of the formula 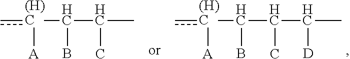
wherein A, B, C, and D independently of one another represent hydrogen or C₁₋₄ alkyl, and
wherein the (alkyl) pyrazine according to formula (Ia) and (Ib) is selected from the group consisting of
5-Methyl-6,7-dihydro-cyclopentapyrazine (corylone pyrazine),
Pyrazine,
2-methylpyrazine,
2,6-Dimethylpyrazine,
2,5-Dimethylpyrazine,
2,3-Dimethylpyrazine,
2,5,6-trimethylpyrazine,
2-Methyl-6,7-dihydro-5H-cyclopenta [b]pyrazine,
2,5-Dimethyl-6,7-dihydro-5H-cyclopenta [b]pyrazine,
5,6,7,8-tetrahydroquinoxaline (cyclohexapyrazine), and
2-Methyl-5,6,7,8-tetrahydroquinoxaline,
2-Ethyl-3-methylpyrazine,
2-Ethyl-3,5-dimethylpyrazine,
2-Ethyl-3,6-dimethylpyrazine,
2-Ethyl-3,5,6-trimethylpyrazine,
2,3-Diethyl-5-methylpyrazine,
2-Ethylpyrazine,
2,5-Diethyl-3,6-dimethylpyrazine,
2,6-Diethyl-3,5-dimethylpyrazine,
2,3-Diethyl-5,6-dimethylpyrazine,
Tetraethylpyrazine, and
2,3-Diethylpyrazine,
wherein said formulation is produced by the process according to claim 27.

49. The method according to claim 44,
wherein the (alkyl) pyrazine has a structure of formula (Ia), 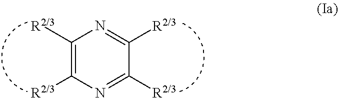
and/or a structure of formula (Ib) 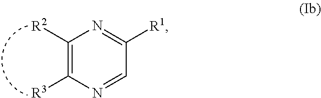
wherein
R¹ represents hydrogen or methyl,
R² and R³ are identical or different and independently of one another represent hydrogen or C₁₋₄
alkyl; or
R² and R³ are bonded to one another and together form a group of the formula 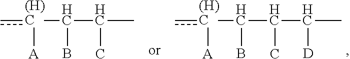
wherein A, B, C, and D independently of one another represent hydrogen or C₁₋₄ alkyl.

50. A method for regulating molar ratios of (alkyl) pyrazines in a process for producing (alkyl) pyrazines, comprising carrying out said process in a solvent comprising at least one hydroxy group,
wherein the (alkyl) pyrazines have the structures of formula (Ia) and formula (Ib), 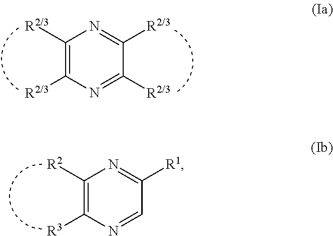
wherein
R¹ represents hydrogen or methyl,
R² and R³ are identical or different and independently of one another represent hydrogen or C₁₋₄ alkyl; or
R² and R³ are bonded to one another and together form a group of the formula 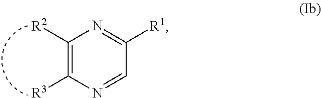
wherein A, B, C, and D independently of one another represent hydrogen or C₁₋₄ alkyl,
wherein the solvent comprising at least one hydroxy group is part of a solvent mixture comprising triacetin, and
the (alkyl) pyrazines are produced according to the method of claim 39.
